(19) United States
(12) Patent Application Publication
(10) Pub. No.: US 2009/0130030 A1
(43) Pub. Date: May 21, 2009

(54) Plural Activating Optical Change Toothpastes, Stimuli and Elements

(76) Inventor: Hans O. Ribi, Hillsborough, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/629,113

(22) PCT Filed: Jun. 10, 2005
(86) PCT No.: PCT/US2005/020702

§ 371 Date: Sep. 22, 2008

Related U.S. Application Data
(60) Provisional application No. 60/579,060, filed on Jun. 10, 2004.

(51) Int. Cl. A61K 8/18

(52) U.S. Cl. 424/49

(57) ABSTRACT

Optical change toothpaste compositions (as well as methods for making and using the same), and methods for inducing and stimulating optical changes and associated elements for imparting optical changes are provided. Optical change toothpastes can possess a plurality of visual and reporting properties and find use in a variety of different applications.

### CROSS-REFERENCE TO RELATED APPLICATIONS

Pursuant to 35 U.S.C. § 119 (e), this application claims priority to the filing date of the U.S. Provisional Patent Application Ser. No. 60/579,060 filed Jun. 10, 2004; the disclosure of which is herein incorporated by reference.

### INTRODUCTION

### Background of the Invention

As is known, inducing children (and adults to some extent) to brush their teeth on a regular basis presents a difficult challenge. The brushing of teeth is perceived as a bothersome necessity by many adults and even more so by children. Insofar as children are concerned, the problem is exacerbated by the fact that children are highly sensitive to bitter tastes, possess a heightened gag reflex and typically utilize an equal amount of toothpaste as adults while having a mouth that is one fourth the size of the adult mouth. Thus, not only is brushing of the teeth an uncomfortable experience for children, but additionally a child's lack of appreciation of the benefits of regular brushing coupled with a child's short attention span renders the twice daily brushing regimen devoid of any positive reinforcement for the typical child.

The availability of a toothpaste or dentifrice which would make brushing more enjoyable for children (and adults) would provide an inducement lacking in existing toothpaste and dentifrice formulations.

As such, there remains a need for an improved toothpaste or dentifrice formulations, particularly for use by children. The present invention satisfies this, and other, needs.

### RELEVANT LITERATURE

See U.S. Pat. Nos. 6,623,382; 6,607,744; 6,564,846; 6,507,989; 6,465,791 6,419,902; 6,046,455; 5,918,981; 5,851,488; 5,798,215; 5,685,641; 5,660,993; 5,622,872; 5,618,735; 4,568,534, and 4,150,106.

### SUMMARY OF THE INVENTION

The subject invention provides optical, e.g., color, change orally acceptable, e.g., toothpaste, compositions and methods for using the same. Color changes in toothpastes can serve multiple purposes. The color change can be utilized as an entertaining and motivating means to encourage children to brush. The color change can serve as a timing mechanism to provide children and parents a simple means to determine when an appropriate brushing duration has been completed. The color change can be used as an indicating means for the diagnosis of an oral dysfunction. The color change can be used as a diagnostic means to help determine whether an individual has a particular disease state expressed in the oral cavity. The color change can be used as a temperature indicating means to help determine if an individual has a fever. The color change can be used as an alternative means for indicating the presence of an oral problem such as plaque buildup. The color change can be sensitized by biochemical means to help serve as an indicator to the presence of tooth decay. The color change can be used as a means to help correct deficiencies in brushing technique. The color change can impart a visual indication as to the timing of another change that can simultaneously be induced in the paste such as a flavor change during brushing.

Activating color change mechanisms and compositions are described for toothpaste whereby toothpaste delivered from a tube exhibits an initial color and changes color during the process of brushing and agitating the toothpaste composition. The optical or color change can take a variety of visual forms. The color can appear from a neutral background color. The color can change from one color to another. The color can initially be bright and then convert to a neutral background color. The color can be emitted in the form of light and can be differentiated from an initial non-emitting background. The color can transition from one state to multiple different sequential colors and the like.

Methods and compositions for inducing the color change can involve but are not limited to: using dye masking; color co-extrusion; chemical; pH; ionic strength changes; enzymatic, and biochemical; mechanical agitation for release; micro-encapsulation dye release during abrasion; physico-chemical, including oxygenation, catalytic, optical illumination, and photochemical; solubilizing and releasing dyes; thermochromic; natural processes; chemical release mechanisms and the like.

Toothpaste matrices can be single component, dual component or multi-component. The complexity of mechanisms used to induce a color change in the toothpaste will dictate the designed approach as to the number of sequestered toothpaste components, sequence of addition, properties during mixing, geometry of toothpaste delivery, and the like.

### DETAILED DESCRIPTION OF REPRESENTATIVE EMBODIMENTS

As summarized above, the present invention provides optical change oral compositions, e.g., toothpastes, dentifrices and the like, and methods for using the same. Also provided are systems and kits that include the subject compositions, e.g., coupled with dispenser and/or applicator, e.g., toothbrush.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Methods recited herein may be carried out in any order of the recited events which is logically possible, as well as the recited order of events.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As summarized above, the subject invention provides oral compositions, particularly toothpaste and related compositions, that include an optical change agent that, in response to an applied stimulus, causes the composition, or at least a portion thereof, to undergo an optical change, such as a visual optical change, e.g., a color change. A feature of many embodiments is that the optical change agent of the composition is an intrinsic optical change component, by which is meant that, in response to the applied stimulus, the agent itself undergoes an optical change, thereby causing the composition as a whole to undergo a color change. As such, the compositions are distinguished from compositions that achieve a color change merely by mixing two different color imparting components to produce a new color that is a blend of the two distinct color imparting components, where the color imparting components do not, themselves, change color and are therefore not intrinsic color change agents. In representative embodiments, the change occurs after a continued duration of applied stimulus, e.g., 2 or more seconds, 10 or more seconds, 30 or more seconds, 1 or more minutes, etc. The compositions may include a single optical change agent or a plurality of such agents, e.g., 2 or more, 3 or more, 4 or more, etc. The compositions and optical change agent(s), alone or in combination, thereof may be responsive to a variety of different applied stimuli (as reviewed in greater detail below) including, but not limited to: mechanical stimulation, electromagnetic radiation, e.g., visible light, temperature change, e.g., heat, etc. Also provided are devices, e.g., toothbrushes, dispensers, etc., configured to apply a stimulus to the compositions of the invention to cause the optical change. In addition, methods of making and using the subject compositions are also provided.

Further features of representative embodiments and aspects of the invention are now reviewed in greater detail.

### Color Change Induction Using Single Component Toothpastes:

Single component toothpastes can be induced to initiate a color change by releasing masked dyes from pigments whereby the masked dye imparts a greater color on the toothpaste matrix than the original pigment dye. By “single component” is meant that the composition includes only one agent that imparts the optical, e.g., color, change to the composition upon application of a stimulus, e.g., brushing time, heat, etc. Single component toothpastes can undergo a color change induction using mechanical/abrasive means whereby an encapsulated dye is released in one colored or non-colored matrix to generate a second color or apparent color that dominates the original color. Single component systems can utilize activation means such as light, oxygenation, or other external means to induce a color change in an initial color paste to a second color.

Single component pastes can utilize an activating additive initially present and latent in the paste whereby mechanical/abrasive means during brushing cause the interaction between the additive or activating agent resulting in a color change from an initial color to a second color. Single component pastes can take advantage of external components on the toothbrush such as a magnet to facilitate a color change such as colored particle migration within the toothpaste to induce an apparent color change during brushing. Alternatively, single component toothpastes can contain a colored component not apparent visually unless optically stimulated by only an appropriate optical light source in the toothbrush being utilized.

Polishing agents used in optical change toothpastes can serve multiple purposes. They can serve to facilitate whitening as intended, be used to induce mechanical stresses and shear on particles comprising an encapsulated dye or dye crystal, they can act as a carrier particle for an optical change agent, they can harbor a charged species for inducing a charge activated color change event, or combinations thereof. Polishing agents include: silicates, borosilicates, fused silicates, inert materials, diatomaceous earth and the like. Polishing agents can be used to facilitate the rupture and release of encapsulated dye compositions. Polishing agents may be present in toothpaste bases from 50% by weight to 0.5%, more usually from 30% to 1%, and typically from 20% to 5%. Polishing agent particle size may range from 1 millimeter to 1 micrometer, more usually from 500 micrometers to 5 micrometers, and typically from 50 micrometers to 10 micrometers.

Standard starting toothpaste ingredients include but are not limited to: sodium fluoride 0.22%, sorbitol (70% solution humectant) 60.00%, silica abrasive 19.00%, polyethylene glycol humectant 5.00, sodium lauryl sulfate surfactant 2.2%, flavor 0.80%, sodium carboxymethyl cellulose binder 0.40%, sodium saccharin sweetener 0.25%, calcium glycerophosphate calcium source 0.01%, trisodium phosphate buffering agent 0.10%, a balance of purified water base balance (depending on desired viscosity and hydrophobic state), and a specified amount of optical agent or activating agent that can be used for affect the optical properties of the optical agent.

Toothpaste compositions can be modified to contain various concentrations of optical change agents or agents capable of stimulating an optical change in the optical change agent (stimulating agent). Optical change agents and stimulating agents can be present in a toothpaste matrix from greater than 50% to as low as 0.01%. More usually the agents will be present at 50% to 0.1%. Typically, the agents will be added at between 25% and 0.5% and most often between 10% and 1%.

The ratio of optical change agent to simulating agent can range from 99.9% optical change agent and 0.1% stimulating agent to 99.9% stimulating agent to 0.1% optical change agent. The exact ratio will depend on the desired interaction between the interacting agent pairs. More usually, the ratio will vary between 99% and 1% of respective agents and more often between 90% and 10% of respective agents.

### Color Change Induction Using Dual and Multi-Component Toothpastes:

Dual component toothpastes take advantage of sequestering an initial dominating color component and a color change induction component. Initially, the two sequestered components can be mixed to a first initial color and then change to an induced second color as the inducing component interacts with the initial dominating color component.

Dual component toothpastes can be used to partition a pH sensitive colored component in one chamber and a pH buffered second component in a second chamber. Upon mixing the initial colorized component can be induced to change color by the changing surrounding pH. Dual component color systems can sequester an ionic component in one chamber which is designated to induce a color change in an ionic strength sensitive component present in the second chamber.

Simplified color change systems can be utilized in dual or multi-component toothpastes whereby the paste extrusion process can be used to obscure one colored component with an outer surrounding second paste component. The toothpaste tube geometry can be adapted such that a small narrow stream of a first component can be shrouded and encased by co-extrusion of an initial dominating paste component. Only mixing will reveal and result in the formation of a new dominating color brought about by exposing the initially obscured inner dye component.

Multi-component pastes can be formulated to create multiple sequential color changes. For example, a pH sensitive color change component can be placed in a sequestered toothpaste tube chamber. A color change agent such as a mildly acidic component can be placed in a second chamber. Another color change agent such as a more basic component can be placed in a third chamber. The components can be formulated to release the acidic or basic formulations sequentially. The acidic component can have an immediate effect on the pH sensitive color change component during initial mixing. The basic component can have a delayed effect whereby a dominating base can initially neutralize the acid and with a specified buffering capacity can change the overall mixed system to a basic condition.

Multiple sequential color changes can be made feasible using various mechanisms for releasing or initiating color changes including: pH, ionic strength, encapsulated dye releasing mechanisms, using sequestered components which break down an encapsulated medium by salvation or other degradation means or the like.

In certain embodiments, a dual component system of the invention is not a composition as described in U.S. Pat. No. 6,419,902.

### Optical Systems Useful for Activating Color Changes:

Chelating metal complexes that are in a non-chelated form exhibit an initial color and transition to a second color when chelated. A variety of different organic chelating compounds or designed caged compounds may find use. The chelation process can take place by placing one member of a chelating agent in one sequestered toothpaste component and a second member in a second toothpaste component. Upon mixing, the chelating complex would be formed resulting in a color change.

Mixing systems can be devised to generate endothermic or exothermic reactions. By way of example, cooling can be accomplished as certain organic materials are dissolved in a compatible solvent. The reaction could be used to stimulate a cooling effect orally as well as to effect a thermochromic change upon cooling a toothpaste component

Dyes indicating pH change can find use in a variety of color change toothpaste formats. Mild buffered acids or bases can be used in combination with a color change pH indicating dye. By way of example, but not limitation, pH-indicating dyes can include several dye types. Chlorophenol red turns yellow at conditions more acidic than pH 5.7, purple if more alkaline than pH 6.4, and brown or grey if the pH falls between 5.8 and 6.4. Cresol red turns yellow at conditions more acidic than pH 7 and red if more alkaline than pH 7.5. Brom thymol blue turns yellow at conditions more acidic than 6.0 and if more alkaline than 6.5. Methyl red turns red at acidic-conditions near 5.0 and turns greenish yellow if more alkaline than 6.0. Other pH sensitive dyes include, but are not limited to Basacryl, X-RL Yellow, Astrazon Blue Frr, Astrazon Brilliant Red 4G, Acid Violet 19, Acid Green 3, Basantol Green 910, Pyranne 120, Acid Red 52, Acid Red 388, xanthene dyes, Acid Red 87. 2,4,5,7-tetrabromo-9-0-carboxylphenyl-6-hydroxy-3-isozanthone, Phloxine B, Acid Red 52, Erythrosine, Erythrosine Bluish, pH sensitive leuco dyes and the like.

The pH of one toothpaste component can be utilized to hold back the color transition of a pH sensitive dye whereas, the buffering capacity and pH level of the second component overcomes the pH level of the first component. During mixing, the final pH change is sufficient to cause the leuco dye to change form a colorless state to a colored state.

In certain embodiments where a pH sensitive dye is used as an optical change agent, at least one non-pH sensitive dye optical change agent is also present. In certain embodiments, the composition does not include a pH sensitive optical change agent. In certain embodiments, the color change agent is not phenolphthalein.

Photochromic dyes (i.e. dyes responsive to an electromagnetic, such as visible light, stimulus) can find use in a variety of color change toothpaste mediums and formats. Photochromic materials can include but are not limited to dyes including: 1,3-Dihydro-1,3,3-trimethylspiro[2H-indole-2,3′-[3H]phenanthr[9,10-b](1,4)oxazine]; bicyclo[2.2.1]hepta—2,5-diene; benzyl viologen dichloride; 4,4′-bipyridyl; 6-bromo-1′,3′-dihydro-1′,3′,3′-trimethyl-8-nitrospiro[2H; 5-chloro-1,3-dihydro-1,3,3-trimethylspiro[2H-indole-2,3′-(3H)naphth[2,1-b](1,4)oxazine]; 6,8-dibromo-1′,3′-dihydro-1′3′,3′-trimethylspiro[2H; 1,1′-diheptyl-4,4′-bipyridinium dibromide; 1′,3′-dihydro-5′-methoxy-1′,3′,3′; 1′,3′-dihydro-8-methoxy-1′,3′3′-trimethyl-6-nitrospiro[2H]; 1′,3′-dihydro-1′3′,3′-trimethyl-6-nitrospiro[2H-1-benzopyran-2,2′-(2H)-indole]; 1,3-dihydro-1,3,3-trimethylspiro[2H-Indole-2,3′-[3H]naphth[2,1-b][1,4]oxazine]; 1,1′-dimethyl-4,4′-bipyridinium dichloride; 5-chloro-1,3-Dihydro-1,3,3-trimethylspiro[2H-indole-2,3′-(3H)phenanthr[9,10-b](1,4)oxazine]; 5-methoxy-1,3,3-trimethylspiro[indoline-2,3′-[3H]naphtho[2,1-b]pyran]; 2,3,3-trimethyl-1-propyl-3H-indolium iodide and the like.

Thermochromic dyes can find use in a variety of color change toothpaste mediums and formats. Thermochromic dyes can include but are not limited to compounds including: bis(2-amino-4-oxo-6-methylpyrimidinium)-tetrachlorocuprate(II); bis(2-amino-4-chloro-6-methylpyrimidinium)hexachlorod-icuprate(II); cobalt chloride; 3,5-dinitro salicylic acid; leuco dyes; spiropyrenes and the like.

Thermochromic dyes (i.e., dyes responsive to a temperature change, e.g., heat, stimulus) can find use in a variety of oral care or oral hygiene applications and formats. Thermochromic dyes can include but are not limited to compounds including: bis(2-amino-4-oxo-6-methylpyrimidinium)-tetrachlorocuprate(II); bis(2-amino-4-chloro-6-methylpyrimidinium)hexachlorod-icuprate(II); cobalt chloride; 3,5-dinitro salicylic acid; leuco dyes; spiropyrenes, bis(2-amino-4-oxo-6-methylpyrimidinium) tetrachlorocuprate(II) and bis(2-amino-4-chloro-6-methylpyrimidinium) hexachlorodicuprate(II), benzo- and naphthopyrans (Chromenes), poly(xylylviologen dibromide, di-beta-naphthospiropyran, Ferrocene-modified bis(spiropyridopyran), isomers of 1-isopropylidene-2-[1-(2-methyl-5-phenyl-3-thienyl)ethylidene]-succinic anhydride and the Photoproduct 7,7adihydro-4,7,7,7a-tetramethyl-2-phenylbenzo[b]thiophene-5,6-dicarboxylic anhydride, and the like.

Other thermochromic dyes of interest include leuco dyes including color to colorless and color to color formulations, sensitive leuco dyes, vinylphenylmethane-leucocyanides and derivatives, fluoran dyes and derivatives, thermochromic pigments, micro and nano-pigments, molybdenum compounds, doped or undoped vanadium dioxide, indolinospirochromenes, melting waxes, encapsulated dyes, liquid crystalline materials, cholesteric liquid crystalline materials, spiropyrans, polybithiophenes, bipyridine materials, microencapsulated, mercury chloride dyes, tin complexes, combination thermochromic/photochromic materials, heat formable materials which change structure based on temperature, natural thermochromic materials such as pigments in beans, various thermochromic inks sold by Securink Corp. (Springfield, Via), Matusui Corp., Liquid Crystal Research Crop., or any acceptable thermochromic materials with the capacity to report a temperature change or can be photo-stimulated and the like. The chromic change agent selected will depend on a number of factors including cost, material loading, color change desired, levels or color hue change, reversibility or irreversibility, stability, and the like.

Alternative thermochromic materials can be utilized including, but not limited to: light-induced metastable state in a thermochromic copper (II) complex Chem. Commun., 2002, (15), 1578-1579 under goes a color change from red to purple for a thermochromic complex, [Cu(dieten)₂](BF4)2 (dieten=N,N-diethylethylenediamine); encapsulated pigmented materials from Omega Engineering Inc.; bis(2-amino-4-oxo-6-methyl-pyrimidinium)-tetrachlorocuprate(II); bis(2-amino-4-chloro-6-methylpyrimidinium)hexachlorod-icuprate(II); cobalt chloride; 3,5-dinitro salicylic acid; leuco dyes; spiropyrenes, bis(2-amino-4-oxo-6-methylpyrimidinium)-tetrachlorocuprate(II); bis(2-amino-4-chloro-6-methylpyridinium) hexachlorod-icuprate(II); cobalt chloride; 3,5-dinitro salicylic acid; leuco dyes; spiropyrenes, bis(2-amino-4-oxo-6-methylpyrimidinium)tetrachlorocuprate(II) and bis(2-amino-4-chloro-6-methylpyrimidinium)hexachlorodicuprate(II), benzo- and naphthopyrans (Chromenes), poly(xylylviologen dibromide, di-beta-naphthospiropyran, Ferrocene-modified bis(spiropyridopyran), isomers of 1-isopropylidene-2-[1-(2-methyl-5-phenyl-3-thienyl)ethylidene]-succinic anhydride and the Photoproduct 7,7adihydro-4,7,7,7a-tetramethyl-2-phenylbenzo[b]thiophene-5,6-dicarboxylic anhydride, and the like. Encapsulated leuco dyes are of interest since they can be easily processed in a variety of formats into a plastic or putty matrix. Liquid crystal materials can be conveniently applied as paints or inks to surfaces of color/shape/memory composites. Photo-luminescent compounds can find use in a variety of color change toothpaste mediums and formats. Photo-luminescent compounds can include but are not limited to a variety of materials. Greens, green blue and violet can be made with alkaline earth aluminates activated by rare earth ions. By way of example, strontium aluminate can be activated using europium (SrAl03:Eu). Visual wavelengths can include: green at 520 nm, blue-green at 505 nm, and blue at 490 nm. Red and orange colors can be generated with are zinc sulfide.

Fluorescent dyes can find use in various color activation toothpaste mediums and formats. Fluorescent dye compounds can include but are not limited to: fluorescein, fluoresceine, resourcinolphthalein, rhodamine, imidazolium cations, pyridoimidazolium cations, dinitrophenyl, tetramethylrhodamine and the like. A wide range of fluorescent dyes that can be activated at various wavelengths and emit light at lower wavelengths can be purchase from Sigma-Aldrich (Saint Louis Mo.) or Molecular Probes (Eugene Oreg.).

Encapsulated food colors can be masked with an opaque encapsulating material, formed in small micro-crystals prior to release, obscured using lake dye pigments, impregnated in opaque waxes or the like. The initially treated dye can be processed such that a physical or chemical means can transform the initial colored state to a second colored state. Typical dyes include: amaranth (Red 2), erythrosine (Red 3), ponceau SX (Red 4), eosine (DC Red 22), phloxine (Red 28), allura red (Red 40), tartrazine (Yellow 5), quinoline yellow SS (DC Yellow 1), sunset yellow FCF (Yellow 6), quinoline yellow WS (DC Yellow 10), fast green FCF (Green 3), alizarine cyanine green F (DC Green 5), quinizarine, green SS (DC Green 6), brilliant blue FCF (Blue 1), indigo, carmine (Blue 2) and the like.

In certain embodiments where a food color is used as an optical change agent, at least one non-food color optical change agent is also present. In certain embodiments, the composition does not include a food color optical change agent. In certain embodiments, the composition includes only a single food color optical change agent.

Various micro-encapsulating polymers and compounding blends can be used for the dye microencapsulation process may include, but are not limited to: methyl cellulose and other cellulose derivatives, polymethyl methacrylate, polymethacrylic acid, polyacrylic acid, polyacrylates, polyacrylamide, polyacryldextran, polyalkyl cyanoacrylate, cellulose acetate, cellulose acetate butyrate, cellulose nitrate, nylon 6, polyterephthalamide and other polyamides, polyvinyl alcohol) polyvinylpyrollidone, shellac, polycaprolactones, polydimethylsiloxanes and other siloxanets, aliphatic and aromatic polyesters, polyethylene oxide, polyethylene-vinyl acetate, polyglycolic acid, polylactic acid and copolymers, poly(methyl vinyl ether/maleic anhydride), polystyrene, polyvinyl acetate phthalate, starch, sol-gels, micro-encapsulating materials used for liquid crystals, micro-encapsulating materials used for thermochromic leuco dyes, micro-encapsulating materials used for photo-chromic dyes, low and high melting waxes such as paraffin, beeswax, carnauba wax, and the like. Various waxes using in cosmetic products may find use. Interlemer polymers (Landec Corporation) may find use.

Dyes can be encapsulated in liquid or dried form within encapsulating materials such as gelatin. Gelatins encapsulations can be clear or opaque. Encapsulating gelatins can be dissolved or ruptured during brushing to release encapsulated dye compositions. Upon rupture or dissolving of the gelatin membrane, concentrated dyes can be released to dramatically change the coloration of the toothpaste during brushing. The timing and degree to which dyes are released from the encapsulation will depend on the encapsulating material composition, its thickness, how easily it is dissolved in the mouth, its mechanical stability, the degree to which the encapsulating material is hydrophobic or hydrophilic, and the like.

Encapsulating liquid dye compositions has the advantage of promoting rapid dye diffusion upon release. The brushing duration indication can therefore be adjusted to appear abruptly. The advantage of solid dye encapsulation is that the dye can more slowly be hydrated and released so that the color progression can appear more slowly and metered. The type and style of color change can therefore be selected base on product requirements and not be hindered by technical limitations.

Components utilized in toothpaste bases such as silicates, diatomaceous earth, other polishing agents and the like can be utilized as carrier and concentrators of a dye composition. Toothpaste particulates can be coated with local high concentrations of dye molecules such that the dye intensity appears low and not obviously visible. Upon brushing the sheer and abrasive forces between the particulate, teeth, and the brush can liberate concentrated dyes and cause a visible color change. Components such as diatomaceous earth are of interest due to their micro-porosity and surface area. Micro-porous particles have the volume and surface capacity to carry large concentrations of dye compared with a solid micro-sphere.

Micro-machined optical and mechanical components, self-assembling micro-components, micro-fluidic components nano-structures, nano-particles, and the like can find use as toothpaste additives. Micro-mechanical, micro-optical, and micro-electronic elements can be designed and utilized as active matrix components that enable new classes of toothpastes. By way of example, micro-mechanical devices can be envisioned to find use to assist in the abrasion process by selectively interacting with plaque deposits on tooth enamel. Micro-optical components can be utilized as an optical communication means for emitting various forms of light intensities, wavelengths, and pulsating patterns. Miniaturized microcircuits can have both reporting and responding characteristics.

Surface and internal characteristics of engineered particles can provide for unique optical responding properties. Micro-fluidic properties can be employed in comprising micro-particles whereby the particles are affected by flow characteristics and the like. Surface characteristics of micro-particles can be designed such that self-assembly of particle arrays can occur. Self-assembly can be employed such that particular shapes or objects can be formed during the brushing and agitation process.

### Optically Changing Toothpastes Stimulated by Interactive Toothbrushes:

Toothpaste can be formulated to interact with design elements in toothbrushes. Toothpaste additives can be selectively included in the toothpaste matrix that are intended to interact with a perturbating element component comprising the toothbrush handle. The optical change in the toothpaste can be stimulated using magnetism, para-magnetism, heating, cooling, mechanical, electrochemical, oxidative or reductive, or by other means to impart the change. The optical change can be initiated by interaction with the toothbrush.

The toothbrush may be designed to have a magnet in the brush head which can selectively interact with magnetic particles comprising the toothpaste. The particles can be paramagnetic or magnetic. The particles can be colored such that they act as a dye-like pigment to color the toothpaste. As brushing occurs, the particles can be made to migrate away from the toothpaste and into the brush head. The effect can be used to generate an apparent color change in the toothpaste.

The toothbrush may be designed to have an optical light source in the handle such that the brush can be used to stimulate an optical effect such as a color effect in the toothpaste that the brush interacts with. The toothpaste can contain a chromic change agent that is selectively optically stimulated by the toothbrush light source. The optical stimulation in the toothpaste can include generating a photochromic effect, a fluorescence excitation effect, a photo-luminescent effect or the like. A wide range of different optical effects can be generated by selecting the appropriate optical stimulation, toothpaste components, and optical stimulating source.

Photochromic materials that can be activated by an illumination source include both monomeric species that undergo and optical change and photo-polymerizable materials whereby a photo-chemical reaction leads to polymerization and color formation (e.g. polydiacetylene formation). Photochromic materials of interest should exhibit observable optical changes at wavelengths utilized by the illumination source.

The toothbrush may be designed to have a thermal heater or heating element in the brush head such that the brush can be used to raise the temperature of the toothpaste during brushing. Toothpastes with a thermochromic agent can be formulated to change response to elevated temperature resulting from the heating toothbrush. Elevated temperatures during brushing can further facilitate improved dental cleansing due to accelerated detergent activity, polishing activity, stain removal, peroxide/whitening activity and the like.

Temperature level in the brush should not exceed temperatures required for comfort. The temperature should be maintained through thermal feedback and kept below scalding temperatures (130° F.). Usually, the thermal heating should be kept between 98° F. and 125° F. Typically, the temperature should be kept in a comfort zone between 100° F. and 110° F. Thermochromic agents compatible with toothpaste compositions should be selected to be within the desired temperature range.

Toothpastes and toothbrushes can be co-engineered such that there is a high selectivity for a formulated color change toothpaste to turn color only through interactivity with a specifically engineered toothbrush. By way of example a sonic toothbrush may be designed to vibrate as a specific frequency that selectively disrupts a certain encapsulated dye in the toothpaste. Conventional toothbrushes or other sonic or vibrating toothbrush would not operate at the vibrational frequencies necessary to cause dye disruption and ultimately a color change in the formulated toothpaste.

### Interactive Toothpaste Dispensers Stimulating Optical Effects in Toothpastes

Toothpaste dispensers can be designed along with toothpaste compositions that, when used together, can be used to create a variety of new and unexpected visual effects in an optical toothpaste. By way of example, a toothpaste dispenser can be equipped with an optical window and an irradiation source. A compatible toothpaste can be formulated with an acceptable optically responsive component such as an irreversible photochromic agent. The toothpaste can travel through and be positioned in the optical window such that a light pulse can be delivered from the dispenser to the surface of the toothpaste. The optical pulse can cause a selective color change directly on the surface of the toothpaste prior to dispensing. Upon dispensing, the toothpaste can be ejected with a defined visible optical imprint on the toothpaste portion.

Using standard photo-masking techniques, words, designs, emblems, messages, figures, graphics, letters, and the like can be patterned on the toothpaste. Creative on-demand messages could be produced. The optical characteristics, output, designs, color variations, attributes and the like can be selected depending on the application of interest.

In another example, toothpaste dispensers can be designed to generate various on-demand color outputs. Fluidic tube designs can be employed along with utilizing selective optical agents and stimulating agents such that a single tube can be used to eject a desired color as specified by the individual using the tube. Fluidic designs can be employed to enable the dispensing of reds, yellow, greens, blues, purples, oranges, browns or any of a variety of other hues. The dispenser/tube can be designed such that the impact to the user can be to simply “dial” the color of interest and eject the designated color using the same tube.

Plural Simultaneous Physiologic Effects Concurrent with Color Changes

An induced color change in toothpaste during brushing can be used in parallel with other property changes such as flavor changes, viscosity changes, texture changes, tartness changes, temperature changes such as cooling or heating, or the like that impart additional physiologic sensations during the brushing process.

The color change can be used as an indication means for releasing medicating agents orally during the brushing process. The plurality of a color change mechanism combined with a medication release mechanism can provide for a simple means for an individual to judge with confidence that a medicating agent was successfully released.

### Chemical Heat Induction in Color Change Toothpastes

Chemical heating can be utilized in combination with optical change toothpastes. The exothermic chemical heating process can be utilized to induce and promote a thermochromic color change in dyes comprising the toothpaste. Likewise, the heating process can assist in promoting improved cleaning due to the elevated temperatures achieved during the chemical heating process. Non-toxic chemical heating processes should be considered. Oxides including calcium oxide can be employed as a chemical heating agent. Various salts can be utilized as the chemical heating agent. Exothermic reactions resulting in heat induction can be generated using various organic and inorganic materials. The chemical heating agent can be partitioned as a material component such that direct exposure is limited or eliminated.

The following examples are offered by way of illustration and not by way of limitation.

### EXPERIMENTAL

### Example 1

### Chemi-Luminescent Light Emitting Toothpaste

A two-component toothpaste can be prepared. The first component can contain fluorescein and phenyl oxalate. The second paste component can contain a hydrogen peroxide solution. When the two components are mixed, a chemi-luminescent reaction can occur whereby visible light can be emitted from the fluorescein present. Mixing at elevated temperatures (body temperature) can increase the illumination intensity. The solution concentrations of fluorescein can be controlled to indicate a one to several minute brushing time.

### Example 2

### pH Change Induction in Two-Component System

A two-component toothpaste can be prepared. The first component can contain Cresol red which turns yellow at conditions more acidic than pH 7. The formula can be adjusted to maintain the pH of the first component at less than pH 7. The second component can contain an acidic-buffered solution at pH 7.5 or above. When the two components are mixed, the red color of the first component will turn yellow when mixing and brushing are adequately accomplished.

### Example 3

### Hydration/Brushing Activated Color Change Toothpaste

Crystalline powderized FD&C food dyes can be coated with a thin coating of carnuba wax. The wax coating can seal the dye crystals making them insoluble in low concentrations of water. The crystal can be dispersed in a single component toothpaste formulation. Formulations containing an abrasive agent can be used to stimulate uncoating and fracturing of the coated dye crystals. During brushing, an initial color of the base toothpaste dominates the paste appearance. Upon brushing and as the wax coating is abrasively removed, the crystalline dye can become exposed to a more aqueous environment and subsequently begin dissolving and dispersing. The dispersing dye can then dominate the overall color resulting in a color change.

### Example 4

### Light Glow/Emitting Toothpaste

A light glow/emitting toothpaste can be formulated using a single component toothpaste composition. A standard translucent toothpaste base is desirable so that good illumination and emission can be accomplished. Photo-luminescent compounds including zinc sulfide or strontium aluminate activated by europium can be mixed directly with the toothpaste composition. Normal room light, sunlight or various electrical light sources can be used to illuminate the pigment. The pigment will emit light for a period of time following the illumination charging effect. The duration of illumination can be used as a means for determining brushing duration.

### Example 5

### Toothbrush with Illumination Source for Activating Light Glow/Emitting Toothpaste

A toothbrush comprising a standard brushing means and an optical illumination means can be prepared using a high intensity light emitting diode as a light source and a battery as a power source. The toothbrush handle and head can be made with a clear polyester material and serve as light guide from an imbedded light source to the bristles and contacting toothpaste. Toothpaste containing a glow/light-emitting component can be illuminated and charged to emit light using the imbedded light source.

### Example 6

### Fluorescence Dye Released and Revealed with Illuminating Toothbrush

A toothpaste with fluorescence activation can be made as a single component toothpaste base. The single component toothpaste can have a fluorescence dye such as fluoresceine, rhodamine, imidazolium, or the like mixed into the toothpaste base. Upon brushing, the fluorescent dye can be dispersed and available for fluorescence excitation using an illumination source. The toothbrush with an illuminating source described in Example 5 above can be constructed with a compatible light source such as a UV light emitting diode (300-400 nanometers) capable of exciting the fluorescence dye. Regulation of timing of releasing the dye can be accomplished using encapsulation process as described in Example 3. The appearance of fluorescence emission during brushing can be used as a means for determining brushing duration.

### Example 7

### Enzyme Activated Color Initiation Toothpaste

Salivary digestive enzymes such as salivary amylase, trypsin and chymotrypsin can be used to break down commensurate encapsulating materials that can be used to mask or encased dyes. The digestive enzymatic process can be used to generate a color change by liberating a concentrated encapsulated dye into the mouth during brushing where the encapsulated dye could be present in the toothpaste. The enzymatic process for dye release could take advantage of natural digestion processes and be used as an indication means for brushing duration.

### Example 8

### Photochromic Color Change Toothpaste

A single component photochromic toothpaste can be made by mixing photochromic agents into the toothpaste base. By way of example, functionalized spiro compounds such as nitrospiro compounds, trimethylspiro compounds, and the like can be used as photochromic agents and added directly to a non-colored toothpaste base. It is desirable to use highly light reactive materials such that a visible color change can be seen as the toothpaste is dispensed from a light shielded toothpaste tube. The color change can be regulated such that the color appearance is rapid or such that the color change occurs more slowly as the toothpaste is being utilized during brushing. The photochromic process can be used as an indication means for determining brushing duration by matching the color change event with a 1-2 minute brushing time.

Illumination sources on a toothbrush as described in Example 5. can further be utilized to induce a photochromic change in a photochromic agent comprising the toothpaste. During brushing, the illumination source on the toothbrush can photo-activate the photochromic agent in the paste. The photochromic agent can change color over time depending on the exposure level. The user can observe the light activated color change and easily judge that the expected brushing time has been completed. The degree to which the color change will be dependent on the type of photochrome utilized, the intensity of the illumination source, the wave length of the illumination source, the concentration of the photochromic agent, temperature, time, the degree to which foaming or other paste constituent block the light and the like.

### Example 9

### Colorized Paramagnetic Micro-Particles that Separate from Toothpaste to Change Paste Color

Colorized paramagnetic microparticles can be added to colorless toothpaste base to generate vivid colors of interest. The particles can be conveniently separated and removed from the toothpaste during brushing by using an adequately strong magnet placed within the toothbrush head. During brushing, the colorized paramagnetic particles become transiently magnetized and migrate to the brush head. The color separation causes an apparent color change to the toothpaste during brushing. The color separation and color change can be used as a means for determining brushing duration by matching the color separation event with a 1-2 minute brushing duration time. The paramagnetic particles can be conveniently removed from the brush head by running faucet water over the head.

### Example 10

### Colorized Particle Agglutination in Toothpaste Causing Apparent Color Change During Mixing

A two component toothpaste can be formulated where one component can comprise a toothpaste base containing colored microparticles with one member of a binding pair. The second toothpaste component can contain a non-colored second member of a binding pair. Mixing of the two toothpaste components can result in the two members of the binding pair to interact and bind. Polyvalent binding can result in the agglutination of the colorized particles. The agglutination reaction acts to scavenge the colored particles and diminish the overall toothpaste color. As agglutination proceeds, small single particles aggregate into fewer larger particles lowering the average color intensity. At completion, few large aggregates remain rendering the overall color nearly completely diminished. The color disappearance can be used as a means for determining brushing duration.

### Example 11

### Colorized Particle Agglutination in Toothpaste Causing Apparent Color Change During Mixing Indicating Analyte Presence

A two component toothpaste can be formulated where one component can comprise a toothpaste base containing colored microparticles with one member of a binding pair. The second toothpaste component can contain a non-colored second member of a binding pair. Mixing of the two toothpaste components can result in the two members of the binding pair to interact and bind. The agglutination process can also be used as an immuno-assay diagnostic approach for testing suspected analytes in saliva. The loss of color can be used as a monitoring means to determine if an analyte is present in an individual's saliva. If a toothpaste were to turn color during brushing, the color loss could be used as a measure of a disease state.

### Example 12

### Color Change Toothpowder

Hydration sensitive and mechanically disruptive micro-encapsulated dyes can be used in single component toothpastes to release dye and cause a color change during brushing with the powder. The powder can initially look white, discolored, off-white or the like. During brushing, water present in saliva and from additional tap water when combined with brushing action against teeth, can cause the dye to be released and result in an apparent color change during brushing. The color change can be used as a means for determining brushing duration.

### Example 13

### Micro-Encapsulated Water Threshold Dyes in Toothpaste that Changes Color on Brushing

Color change toothpastes using dye developers used in pressure sensitive and thermal printing process can be utilized. A resin containing or encapsulating a dye can be prepared. The resin can be formulated with a color formation dye such as crystal violet lactone. Activators can be used to initiate color formation from an initial colorless state to a deeply colored state. Water infusion resulting from increased water content from oral contact, tap water, or from a separate toothpaste component can be used to initiate the color change reaction. Diffusion of the dye out of the encapsulating matrix can be used to generate a vivid color during brushing.

Alternative examples can include but are not limited to: rupturing dye sacks for release of dye from an initial color to a second color; abrasion induced encapsulated dye exposure; body temperature induced encapsulated dye rupture; the use of natural dye pigment color change induction; the use of thermal induction in glow in the dark pigments; dye unbinding during mechanical disruption; crystal disruption induced dye release; ionic charge change induced color change; autocatalytic color change induction; caged complex and chelating induced color change; alternative wax encapsulated dyes released by pressure and heat; cosmetic based encapsulated composition, which changes color with heat/friction; natural floral and leaf pigments that are induced to change color; co-extruding toothpaste tube where thick outer toothpaste ring hides intensely colored inner core; anisotropic dye alignment for one color appearance followed by disruption to color change isotropic phase and other related physical, chemical, optical, physiologic means for inducing color changes in toothpastes.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A toothpaste composition comprising an intrinsic optical change component that changes optical properties in response to an applied stimulus, wherein when said composition includes a pH sensitive optical change component, said composition includes at least one additional non-pH sensitive optical change component.

2. The composition according to claim 1, wherein said composition does not include a food color optical change component.

3. The composition according to claim 1, wherein said composition includes chelating metal complex optical change component.

4. The composition according to claim 1, wherein said composition includes a photochromic optical change component.

5. The composition according to claim 1, wherein said composition includes a thermochromic dye optical change component.

6. The composition according to claim 1, wherein said composition includes a photo-luminescent optical change component.

7. The composition according to claim 1, wherein said composition includes a fluorescent optical change component.

8. The composition according to claim 1, wherein said composition includes a pH sensitive dye.

9. The composition according to claim 1, wherein said composition includes an encapsulated release dye as a optical change component.

10. The composition according to claim 1, wherein said applied stimulus is a mechanical stimulus.

11. The composition according to claim 10, wherein said mechanical stimulus is applied to said composition by a toothbrush.

12. The composition according to claim 1, wherein said applied stimulus is light.

13. The composition according to claim 12, wherein light is applied to said composition by a toothbrush.

14. The composition according to claim 12, wherein light is applied to said composition by a dispenser.

15. The composition according to claim 1, wherein said stimulus is a temperature change.

16. The composition according to claim 15, wherein said temperature change is heat.

17. The composition according to claim 15, wherein temperature change is applied to said composition by a toothbrush.

18. The composition according to claim 15, wherein temperature change is applied to said composition by a dispenser.

19. The composition according to claim 1, wherein said composition undergoes two or more optical changes in response to at least one applied stimulus.

20. The composition according to claim 1, wherein said composition undergoes a physical change in response to an applied stimulus.

21. The composition according to claim 1, wherein timing of said optical change is dependent on duration of said applied stimulus.

22. The composition according to claim 1, wherein said composition comprises a single optical change component.

23. The composition according to claim 1, wherein said composition includes at least two optical change components.

24. A device configured to be used with a composition according to claim 1, wherein said device is capable of exerting an applied stimulus to said composition to cause said composition to undergo an optical change.

25. The device according to claim 24, wherein said device is a toothbrush.

26. The device according to claim 25, wherein said device is a holder for said composition.

27. The device according to claim 26, wherein said holder is a dispenser for said composition.

28. The device according to claim 1, wherein said device is in contact with said composition.

29. A system comprising:
a device according to claim 24; and
a composition comprising an intrinsic optical change component that changes optical properties in response to an applied stimulus, wherein when said composition includes a pH sensitive optical change component, said composition includes at least one additional non-pH sensitive optical change component.

30. A method of making a toothpaste composition that exhibits an optical change during brushing, said method comprising:
combining an intrinsic optical change agent with at least one additional toothpaste component to produce said toothpaste composition, wherein when said composition includes a single optical change agent, said agent is not a pH sensitive dye.

31. In a method of brushing teeth, the improvement comprising employing a toothpaste composition according to claim 1.
